(19) United States
(12) Patent Application Publication
(10) Pub. No.: US 2023/0207065 A1
(43) Pub. Date: Jun. 29, 2023

(54) AUTOMATED PATHOGENIC MUTATION CLASSIFIER AND CLASSIFICATION METHOD THEREOF

(71) Applicant: National Yang Ming Chiao Tung University, Hsinchu City (TW)

(72) Inventors: Jui-Hung HUNG, Hsinchu City (TW); Wei-Chen CHANG, Taichung City (TW)

(21) Appl. No.: 18/058,767

(22) Filed: Nov. 24, 2022

(30) Foreign Application Priority Data
Dec. 23, 2021 (TW) 110148492

(51) Int. Cl. G16B 40/00; G16H 50/70; G16H 50/80; G16B 20/20; G16B 20/40; G16B 25/10

(52) U.S. Cl. CPC G16B 40/00; G16H 50/70; G16H 50/80; G16B 20/20; G16B 20/40; G16B 25/10

(57) ABSTRACT

An automated pathogenic mutation classification method is provided, which includes producing a population score using a population database based on related information. A variant type score is produced using a variation pattern prediction tool based on the related information. A clinical score is produced using the related information or a clinical database based on the related information. A functional score is produced using a functional variant hazard prediction tool based on the related information. The population score, the variant type score, the clinical score, and the functional score are summed to obtain a pathogenic score. Probability that mutation sites suffer from a corresponding disease is determined based on the pathogenic score. When the pathogenic score is higher, the probability of the mutation sites suffering from the corresponding disease is higher.

### CROSS-REFERENCE TO RELATED APPLICATION

This application claims priority to Taiwan Application Serial Number 110148492, filed on Dec. 23, 2021, which is herein incorporated by reference.

### BACKGROUND

### Field of Invention

The present disclosure relates to a classifier and a classification method, and more particularly, to an automated pathogenic mutation classifier and a classification method thereof.

### Description of Related Art

In 2013, the American College of Medical Genetics and Genomics (ACMG) and the Association for Molecular Pathology (AMP) jointly proposed a set of guidelines, which was formulated by collecting common ways of judging variants at that time, and then integrating these ways into consideration. The guidelines can be applied clinically to various genes, and the guidelines are recommended for identifying variants associated with Mendelian disorders. However, the guidelines have many shortcomings, such as unclear definition, inconsistent interpretations by different units, and the way of judging results, which make it difficult to expand the guidelines.

In order to improve the shortcomings of ACMG, Sherloc rules were proposed. Sherloc rules collected more than 4,000 variants, classified them based on ACMG-AMP, and added 108 items. However, in practice, Sherloc rules are imprecise in classification of specific diseases in order to serve as general guidelines. Many guidelines require human judgment, and thus are difficult to achieve full automation. Therefore, how to realize fully automatic determination of pathogenic mutation sites, the existing technology needs to be improved.

### SUMMARY

An embodiment of the present disclosure provides an automated pathogenic mutation classification method, which includes: receiving related information, the related information including variant sequence information, the variant sequence information including patient information and variant analysis, patient's family information and variant analysis, or unrelated person information and variant analysis; producing a population score using a population database based on the related information, in which the population database includes genome aggregation database (gnomAD), 1,000 genomes project database, Clinvar database or a combination thereof; producing a variant type score using a variation pattern prediction tool based on the related information; producing a clinical score using the related information or a clinical database based on the related information, in which the clinical database includes Clinvar database; producing a functional score using a functional variant hazard prediction tool based on the related information; summing the population score, the variant type score, the clinical score, and the functional score to produce a pathogenic score; and determining probability that a plurality of mutation sites in the variant sequence information suffer from a corresponding disease based on the pathogenic score, in which when the pathogenic score is higher, the probability of the mutation sites suffering from the corresponding disease is higher.

In some embodiments, the related information further includes loss-of-function test data, protease kinetic chemical analysis data, special target disease or gene selection, or a combination thereof.

In some embodiments, the step of using the population database includes: performing a frequency variation analysis on the variant sequence information using the population database to generate a first population score; performing a homozygous observational analysis on the variant sequence information using the population database to generate a second population score; and summing the first population score and the second population score to obtain the population score.

In some embodiments, the population database includes genome aggregation database (gnomAD) and 1,000 genomes project database, in which the step of performing the frequency variation analysis on the variant sequence information using the population database, when the mutation sites in the variant sequence information in a plurality of alleles in the genome aggregation database are greater than a predetermined threshold number, continue to use the genome aggregation database to perform the frequency variation analysis; or when the mutation sites in the variant sequence information in the alleles in the genome aggregation database are less than or equal to the predetermined threshold number, use the 1,000 genomes project database to perform the frequency variation analysis.

In some embodiments, the step of performing the homozygous observational analysis on the variant sequence information using the population database, when the mutation sites in the variant sequence information in a plurality of alleles in the genome aggregation database are greater than a predetermined threshold number, continue to use the population database to perform the homozygous observational analysis; or when the mutation sites in the variant sequence information in the alleles in the genome aggregation database are less than or equal to the predetermined threshold number, the homozygous observational analysis is not performed.

In some embodiments, the step of producing the variant type score using the variation pattern prediction tool based on the related information includes: producing gene sequence variation hazard information using the variation pattern prediction tool based on the related information; the gene sequence variation hazard information including variant type data and a gene loss-of-function index; and performing a null variant analysis, a splice variant analysis, a missense variant analysis, an in-frame indels variant analysis, a start loss variant analysis, a silent variant analysis, an intronic variant analysis, a non-coding variant analysis in UTR or promoter, a copy-number variation (CNV) analysis, or a combination thereof based on the variant type data to obtain the variant type score.

In some embodiments, the gene loss-of-function index is probability of loss of function intolerance.

In some embodiments, when the null variant analysis, the splice variant analysis, and the start loss variant analysis are performed to evaluate the probability of loss of function intolerance, if the probability of loss of function intolerance is greater than a predetermined threshold, it automatically determines that a risk is high when one or more genes in the related information are loss of function; or if the probability of loss of function intolerance is less than the predetermined threshold, it automatically determines that a risk is low when one or more genes in the related information are loss of function.

In some embodiments, the step of producing the clinical score using the related information or a clinical database based on the related information includes judging whether it is a patient based on the related information, and then performing a dominant-recessive analysis, a genotype analysis, a cis-trans analysis, a disease penetrance analysis, an age of onset analysis, or a combination thereof.

In some embodiments, the functional variant hazard prediction tool includes a scale-invariant feature transform (SIFT) unit, a polymorphism phenotype analysis unit, and a site hazard prediction unit; in which the step of producing the functional score using the functional variant hazard prediction tool based on the related information includes judging whether the mutation sites in the variant sequence information of the related information are a missense variant or a splicing variant, when the mutation sites are the missense variant, analysis with the scale-invariant feature transform unit and the polymorphism phenotype analysis unit is performed to produce the functional score; or when the mutation sites are the splicing variant, analysis with the site hazard prediction unit is performed to produce the functional score.

In some embodiments, in which a report is outputted and the report is the probability of the mutation sites suffering from the corresponding disease based on the pathogenic score.

Another embodiment of the present disclosure provides an automated pathogenic mutation classifier, which includes a computer processor and a memory, the memory storing a plurality of computer program instructions that, when executed by the computer processor, cause the computer processor to implement following steps, including: accessing related information, the related information including variant sequence information, the variant sequence information including patient information and variant analysis, patient's family information and variant analysis, or unrelated person information and variant analysis; producing a population score using a population database based on the related information, in which the population database includes genome aggregation database, 1,000 genomes project database, Clinvar database or a combination thereof; producing a variant type score using a variation pattern prediction tool based on the related information; producing a clinical score using the related information or a clinical database based on the related information, in which the clinical database includes Clinvar database; producing a functional score using a functional variant hazard prediction tool based on the related information; summing the population score, the variant type score, the clinical score, and the functional score to produce a pathogenic score; and determining probability that a plurality of mutation sites in the variant sequence information suffer from a corresponding disease based on the pathogenic score, in which when the pathogenic score is higher, the probability of the mutation sites suffering from the corresponding disease is higher.

In some embodiments, the related information further includes loss-of-function test data, protease kinetic chemical analysis data, special target disease or gene selection, or a combination thereof.

In some embodiments, the step of using the population database includes: performing a frequency variation analysis on the variant sequence information using the population database to generate a first population score; performing a homozygous observational analysis on the variant sequence information using the population database to generate a second population score; and summing the first population score and the second population score to obtain the population score.

In some embodiments, the population database includes genome aggregation database and 1,000 genomes project database, and the step of performing the frequency variation analysis on the variant sequence information using the population database, when the mutation sites in the variant sequence information in a plurality of alleles in the genome aggregation database are greater than a predetermined threshold number, continue to use the genome aggregation database to perform the frequency variation analysis; or when the mutation sites in the variant sequence information in the alleles in the genome aggregation database are less than or equal to the predetermined threshold number, use the 1,000 genomes project database to perform the frequency variation analysis.

In some embodiments, the step of performing the homozygous observational analysis on the variant sequence information using the population database, when the mutation sites in the variant sequence information in a plurality of alleles in the genome aggregation database are greater than a predetermined threshold number, continue to use the population database to perform the homozygous observational analysis; or when the mutation sites in the variant sequence information in the alleles in the genome aggregation database are less than or equal to the predetermined threshold number, the homozygous observational analysis is not performed.

In some embodiments, the step of producing the variant type score using the variation pattern prediction tool based on the related information includes: producing gene sequence variation hazard information using the variation pattern prediction tool based on the related information; the gene sequence variation hazard information including variant type data and a gene loss-of-function index; and performing a null variant analysis, a splice variant analysis, a missense variant analysis, an in-frame indels variant analysis, a start loss variant analysis, a silent variant analysis, an intronic variant analysis, a non-coding variant analysis in UTR or promoter, a copy-number variation analysis, or a combination thereof based on the variant type data to obtain the variant type score.

In some embodiments, the gene loss-of-function index is probability of loss of function intolerance.

In some embodiments, when the null variant analysis, the splice variant analysis, and the start loss variant analysis are performed to evaluate the probability of loss of function intolerance, if the probability of loss of function intolerance is greater than a predetermined threshold, it automatically determines that a risk is high when one or more genes in the related information are loss of function; or if the probability of loss of function intolerance is less than the predetermined threshold, it automatically determines that a risk is low when one or more genes in the related information are loss of function.

In some embodiments, the step of producing the clinical score using the related information or a clinical database based on the related information includes judging whether it is a patient based on the related information, and then performing a dominant-recessive analysis, a genotype analysis, a cis-trans analysis, a disease penetrance analysis, an age of onset analysis, or a combination thereof.

In some embodiments, in which the functional variant hazard prediction tool includes a scale-invariant feature transform unit, a polymorphism phenotype analysis unit, and a site hazard prediction unit; in which the step of producing the functional score using the functional variant hazard prediction tool based on the related information includes judging whether the mutation sites in the variant sequence information of the related information are a missense variant or a splicing variant, when the mutation sites are the missense variant, analysis with the scale-invariant feature transform unit and the polymorphism phenotype analysis unit is performed to produce the functional score; or when the mutation sites are the splicing variant, analysis with the site hazard prediction unit is performed to produce the functional score.

In some embodiments, the automated pathogenic mutation classifier further comprises an output module connected to the computer processor, and the output module receives the pathogenic score and outputs a report which is the probability of the mutation sites suffering from the corresponding disease based on the pathogenic score.

### BRIEF DESCRIPTION OF THE DRAWINGS

Various aspects of the present disclosure will be most easily understood when the following detailed description is read in conjunction with the accompanying drawings. It should be noted that according to industry standard operating procedures, various characteristic structures may not be drawn to scale. In fact, for clarity of discussion, the size of various characteristic structures may be arbitrarily increased or decreased. In order to make the above and other objectives, features, advantages and embodiments of the present invention easier to understand, the accompanying drawings are described as follows:

FIG. 1 is a flowchart illustrating an automated pathogenic mutation classification method according to some embodiments of the present disclosure;

FIG. 2A is a flowchart of a frequency variation analysis according to some embodiments of the present disclosure;

FIG. 2B is a flowchart of the process A of FIG. 2A;

FIG. 3 is a flowchart of a homozygous observational analysis according to some embodiments of the present disclosure;

FIG. 4 is a flowchart of a null variant analysis according to some embodiments of the present disclosure;

FIG. 5 is a flowchart of a splice variant analysis according to some embodiments of the present disclosure;

FIG. 6 is a flowchart of a missense variant analysis according to some embodiments of the present disclosure;

FIG. 7 is a flowchart of an in-frame indels variant analysis according to some embodiments of the present disclosure;

FIG. 8 is a flowchart of a start loss variant analysis according to some embodiments of the present disclosure;

FIG. 9 is a flowchart of a silent variant analysis according to some embodiments of the present disclosure;

FIG. 10 is a flowchart of an intronic variant analysis according to some embodiments of the present disclosure;

FIG. 11 is a flowchart of a non-coding variant analysis in UTR or promoter according to some embodiments of the present disclosure;

FIG. 12 is a flowchart of a copy-number variation analysis according to some embodiments of the present disclosure;

FIG. 13 is a flowchart of an experimental data analysis according to some embodiments of the present disclosure;

FIG. 14A is a flowchart of clinical data comparison of a patient with unknown pathogenic cause according to some embodiments of the present disclosure;

FIG. 14B is a flowchart of the process B of FIG. 14A;

FIG. 14C is a flowchart of the process C of FIG. 14A;

FIG. 15 is a flowchart of the isolation analysis of FIG. 14C;

FIG. 16 is a flowchart of clinical data comparison of patients with known pathogenic cause according to some embodiments of the present disclosure;

FIG. 17 is a flowchart of clinical data comparison of a healthy subject according to some embodiments of the present disclosure; and

FIG. 18 is a flowchart of a functional predictive analysis according to some embodiments of the present disclosure.

### DETAILED DESCRIPTION

In order to make the description of the present disclosure more detailed and complete, the following provides an illustrative description for implementation aspects and specific embodiments of the present disclosure. However, this is not the only form of implementing or using specific embodiments of the present disclosure. The embodiments disclosed below can be combined or substituted with each other under beneficial circumstances, and other embodiments can also be added to one embodiment without further description or explanation. In the following description, numerous specific details are set forth in detail to enable the reader to fully understand the following embodiments. However, embodiments of the present disclosure may be practiced without these specific details.

In this article, unless the context of the article is specifically limited, otherwise “a” and “the” can refer to a single one or a plurality of. It will be further understood that the terms “comprising”, “including”, “having” and similar terms used herein designate the recited features, regions, integers, steps, operations, elements and/or components, but do not exclude its stated or additional one or more other features, regions, integers, steps, operations, elements, components, and/or groups thereof.

An automated pathogenic mutation classifier of the present disclosure, hereinafter referred to as Holmes, after inputting related information, mainly uses four steps of Sherloc rules: population data, variant type data, clinical data, and functional data to perform fully automated interpretation.

In this article, “related information” may include, but is not limited to, variant sequence information (e.g., patient disease information and variant analysis, patient's family information and variant analysis, unrelated person information and variant analysis (e.g., disease, disease-free, historical analysis records, etc.)), loss of function (LOF) experimental data, protease kinetic chemical analysis data, special target disease or gene selection, or a combination thereof, etc.; or the above data saved in variant call format (VCF); or the above VCF saved in a json file.

In this article, “variant effect predictor (VEP)” is a variant annotation tool with two functions: 1. hazard prediction analysis of gene sequence variation, and 2. hazard prediction analysis for functional aspects caused by gene sequence variation.

In this article, “population database” includes genome aggregation database (gnomAD), 1,000 genomes project database, Clinvar database, exome aggregation consortium (ExAC) database, or a combination thereof.

The VCF file includes but is not limited to the following information: CHROM: reference sequence name; POS: mutated position; ID: code of mutation site; REF: allele of reference sequence; ALT: allele of mutation site; QUAL: quality of mutation site, the larger the value, the greater the probability that the site is the mutation site; FILTER: whether a secondary site should be filtered out; INFO: related information of mutation site; FORMAT: format of mutation site, such as GT:AD:DP:GQ:PL. The json file includes database: it is the path of all databases, and the database generated by itself needs to be saved in a preset way; disease: disease-related data, when the disease data is generated by Omim database, a disease list will be generated, the user can collect the disease's dominant-recessive, early or late onset, severity, penetrance, and gene-related information from the above or by himself as a reference; observation: the vcf path of unrelated patients, which can be provided in plural; patient: provide related information of the patient and the relatives; VEP: provide the path to the VEP execution file.

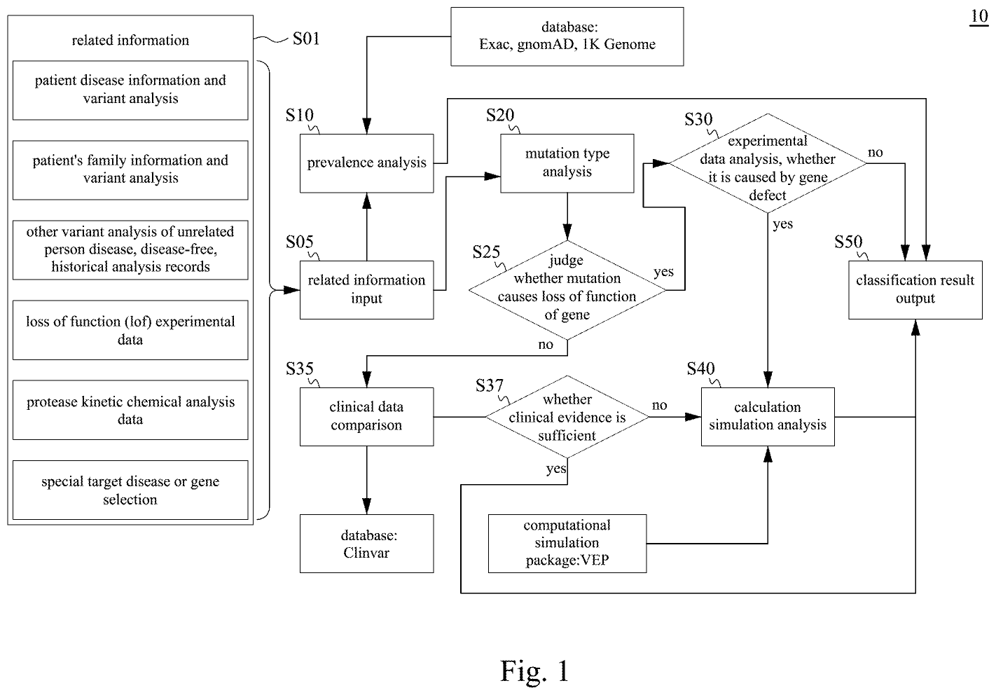

Please refer to FIG. 1. FIG. 1 is a flowchart illustrating an automated pathogenic mutation classification method according to some embodiments of the present disclosure. The classification method 10 includes step S01 of preparing related information, step S05 of inputting related information, step S10 of prevalence analysis, step S20 of mutation type analysis, step S25 of judging whether mutation causes loss of function of gene, and step S30 of experimental data analysis, whether it is caused by gene defect, step S35 of clinical data comparison, step S37 of whether clinical evidence is sufficient, step S40 of calculation simulation analysis, step S50 of classification result output clinical data comparison calculation simulation analysis. The method of scoring of the classifier of the present disclosure is to pass VCF through step S10 to step S50, depending on the judgment process to determine which step to proceed, and each mutation site gets a corresponding evidence score in each step, and finally scores are summed up to obtain a result of the site for pathogenic classification. The score is in line with the evidence and score, and the score of 1≥X>2 is benign, and the score of 2≥X>3 is probably benign, and the score of 3≥X>4 is uncertain, and the score of 4≥X>5 is likely to be pathogenic, and the score of X≥5 is pathogenic. The process is the actual operation process of Holmes, and the user does not need to participate in it.

Specifically, step S05 of receiving related information, the related information includes variant sequence information (e.g., patient disease information and variant analysis, patient's family information and variant analysis, unrelated person information and variant analysis, disease, disease-free, historical analysis records), loss of function (LOF) experimental data, protease kinetic chemical analysis data, special target disease or gene selection, or a combination thereof, etc. Next, proceeds to step S10 and step S20. Step S10 of prevalence analysis, a population score is produced using a population database based on the related information. Step S20 of mutation type analysis, a variant type score is produced using a variation pattern prediction tool based on the related information, in which the variation pattern prediction tool includes, but is not limited to, the variant effect predictor (VEP) for determining the mutation type, i.e., the hazard prediction analysis for gene sequence variation using the variant effect predictor (VEP). Next, proceeds to step S25, whether mutation causes loss of function of gene is judged. If yes, proceeds to step S30, and if no, proceeds to step S35. Step S30 of experimental data analysis, whether it is caused by gene defect, if yes, proceeds to step S40, and if no, proceeds to step S50. Step S35 of clinical data comparison, a clinical score is produced using the related information or a clinical database based on the related information, in which the clinical database includes Clinvar database. Step S37, strength of the clinical evidence is judged. If there is no clinical evidence or insufficient, proceeds to step S40; if the clinical evidence is sufficient, proceeds to step S50. Step S40 of calculation simulation analysis, a functional score is produced using a functional variant hazard prediction tool based on the related information, in which the functional variant hazard prediction tool includes, but is not limited to, the hazard prediction analysis in the variant effect predictor (VEP) for the functional aspects caused by the gene sequence variation. Step S50 of outputting classification, the population score, the variant type score, the clinical score and the functional score are summed to produce a pathogenic score; next, according to the pathogenic score, probability that the mutation sites in the variant sequence information suffer from a corresponding disease is determined based on the pathogenic score. When the pathogenic score is higher, the probability of the mutation sites suffering from the corresponding disease is higher. The detailed steps of step S10 to step S40 will be sequentially described below.

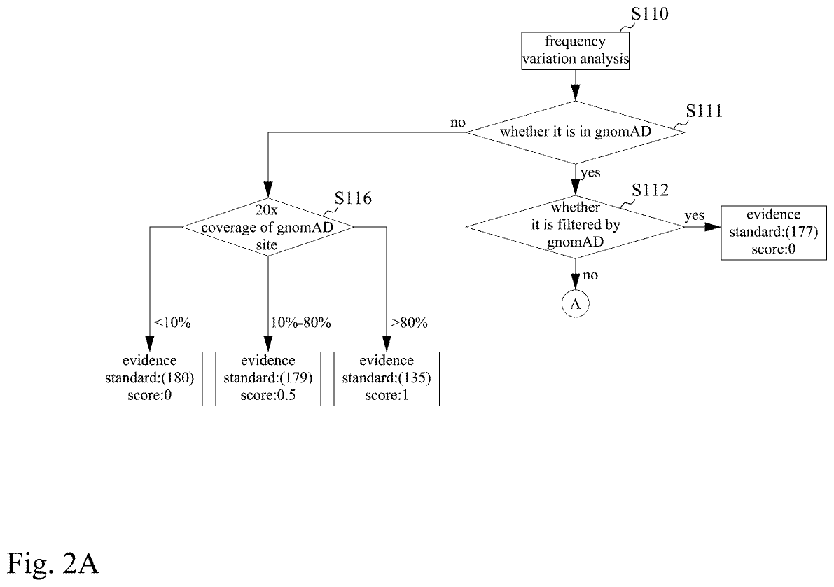

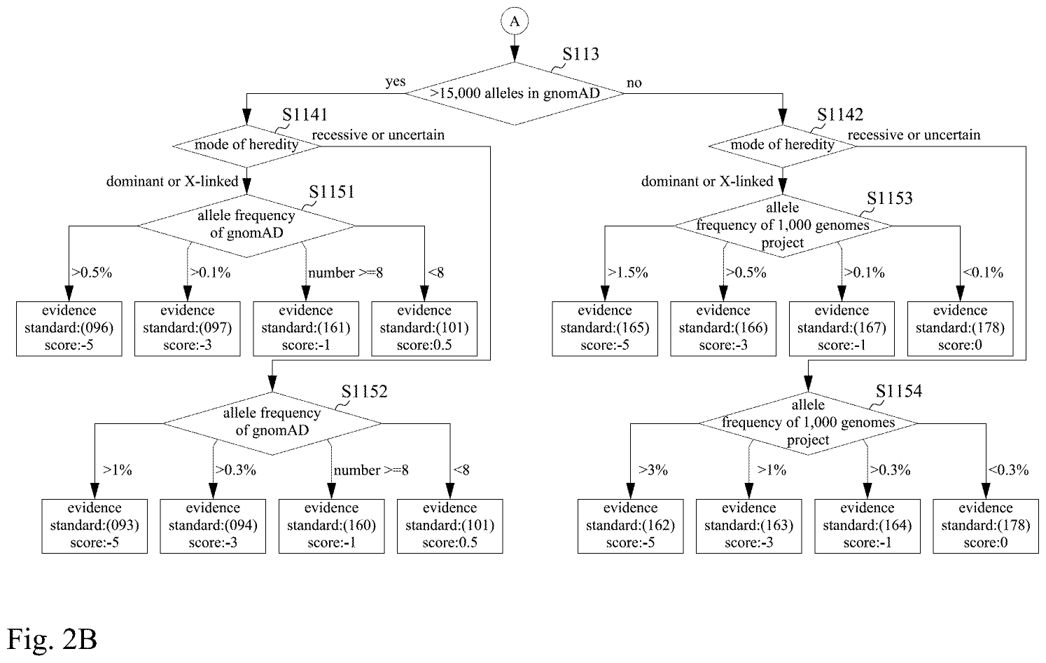

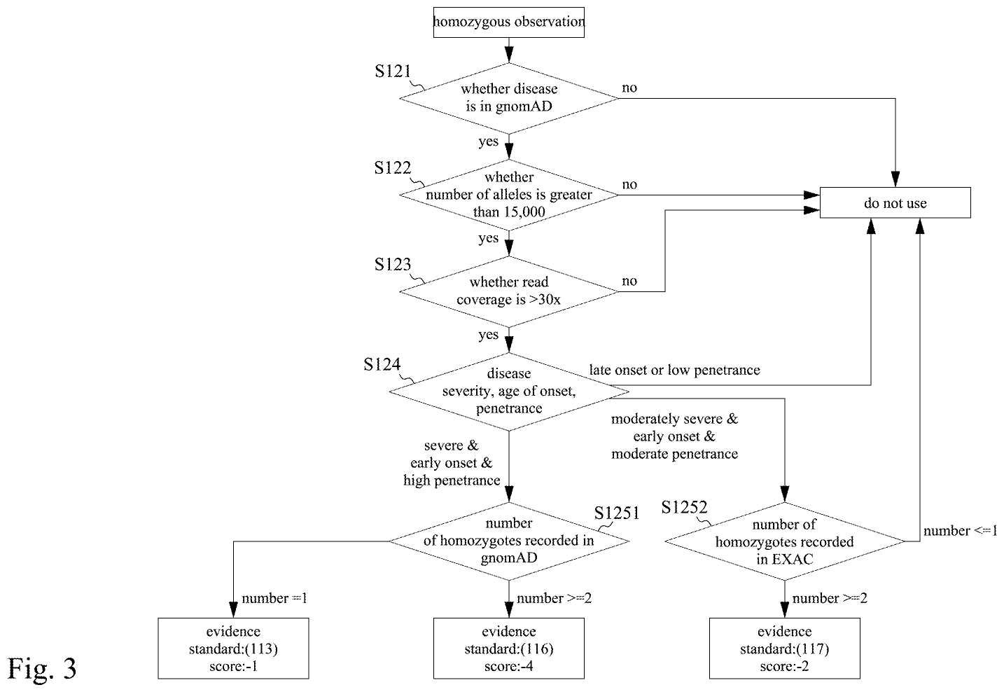

Please refer to FIG. 2A, FIG. 2B and FIG. 3. FIG. 2A is a flowchart of a frequency variation analysis according to some embodiments of the present disclosure, and FIG. 2B is a flowchart of the process A of FIG. 2A, and FIG. 3 is a flowchart of a homozygous observational analysis according to some embodiments of the present disclosure. Step S10 of prevalence analysis includes step S110 of frequency variation analysis and step S120 of homozygous observational analysis. In terms of the frequency variation analysis, Sherloc believed that ACMG-AMP's standard of 5% or more to be benign is too high, and the analysis showed that in a set of 79 disease genes (39 dominant, 40 recessive, 1508 total variants), 97.3% of pathogenic variants had allele frequency of less than 1%, and 94% thereof had 8 alleles or less, so it set more pathogenic scores for classification according to dominant or recessive genotype. In terms of the homozygous observational analysis, when a severe (life-threatening) mutation was found in a homozygous form, Sherloc rules considered it to be reasonably suspected to be a benign mutation, so Sherloc added a score for this part.

Please refer to FIG. 2A and FIG. 2B, step S110 of frequency variation analysis is performed on the variant sequence information using genome aggregation database or 1,000 genomes project database to generate a first population score. Specifically,

in step S111, Holmes first checks whether the site is in gnomAD, if yes, proceeds to step S112, otherwise proceeds to step S116;

in step S112, Holmes confirms whether the site is filtered by gnomAD, if yes, evidence standard is 177 and the first population score is 0, otherwise proceeds to step S113;

in step S113, Holmes confirms whether a number of alleles at the site in gnomAD is greater than a predetermined threshold number (e.g., 15,000, or is used to control for sufficient statistical significance for this database), if yes, proceeds to step S1141 of using gnomAD continuously, otherwise proceeds to step S1142 of using 1,000 genomes project database;

in step S1141, step S1142, Holmes judges dominant or recessive through the file input by the user, if not provided, automatically queries Clinvar sites, and there is no data default recessive; in step S1141, if it is dominant or X-linked, proceeds to step S1151, if it is recessive or uncertain, proceeds to step S1152; in step S1142, if it is dominant or X-linked, proceeds to step S1153, if it is recessive or uncertain, proceeds to step S1154;

in steps S1151, S1152, S1153, and S1154, combined with allele frequency and allele number of gnomAD or 1,000 genomes project database, a corresponding evidence score, that is, the first population score is obtained;

in step S116, if the site is not recorded by gnomAD, Holmes queries a 20X value of the site in a coverage database of gnomAD, and then obtains a corresponding score according to this value, that is, the first population score.

Please refer to FIG. 3, step S120 of homozygous observational analysis is performed on the variant sequence information using genome aggregation database and Clinvar database to generate a second population score; and the first population score and the second population score are summed to obtain the population score. Specifically,

in step S121, Holmes first checks whether the site is in gnomAD, if yes, proceeds to the next step, otherwise it is not used and the second population score is 0;

in step S122, Holmes confirms whether a number of alleles at the site in gnomAD is greater than a predetermined threshold number (e.g., 15,000, or is used to control for sufficient statistical significance for this database), if yes, proceeds to the next step, otherwise it is not used and the second population score is 0;

in step S123, Holmes then judges whether average coverage reaches 30X from the coverage database of gnomAD, if yes, proceeds to the next step, otherwise it is not used and the second population score is 0;

in step S124, Holmes then classifies according to disease severity, penetrance, and age of onset obtained by querying the json file input by the user or automatically querying Clinvar. If it is severe, early onset, and high penetrance, proceeds to step S1251; if it is moderately severe, early onset and moderate penetrance, proceeds to step S1252; if it is moderately severe, early onset and moderate penetrance, it is not used and the second population score is 0;

in step S1251, Holmes determines a number of homozygotes from gnomAD to obtain the second population score;

in step S1252, Holmes determines a number of homozygotes from ExAC to obtain the second population score.

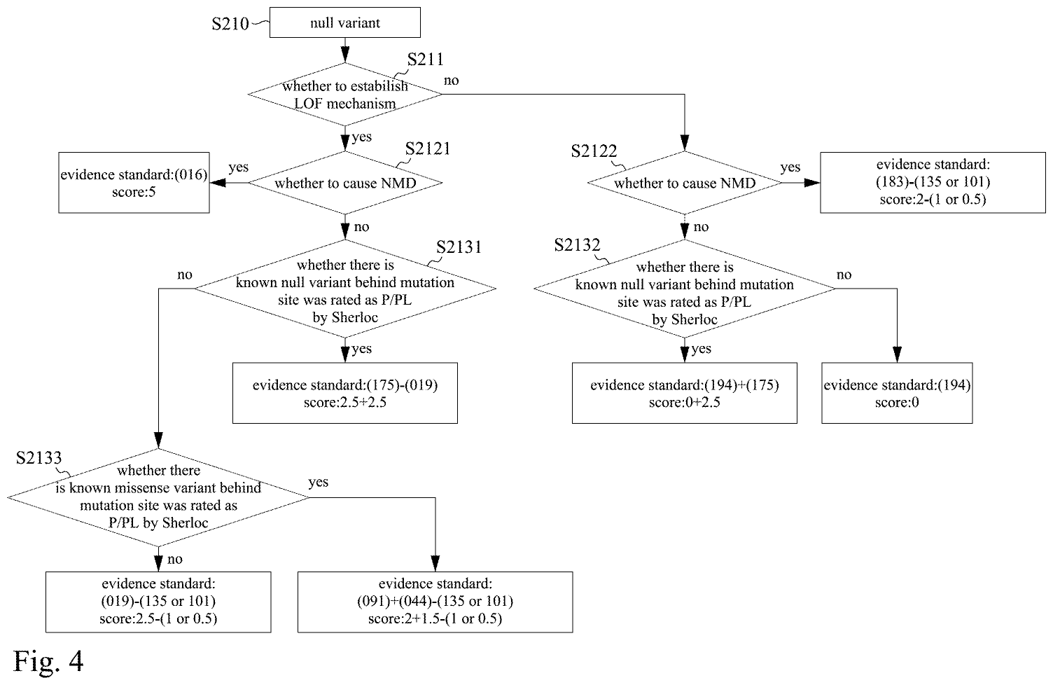

Please refer to FIG. 4 to FIG. 12, step S20 of mutation type analysis, Holmes determines the variant type of the VEP, and then proceeds to step S210 of null variant analysis, step S220 of splice variant analysis, step S230 of missense variant analysis, step S240 of in-frame indels variant analysis, step S250 of start loss variant analysis, step S260 of silent variant analysis, step S270 of intronic variant analysis, step S280 of non-coding variant analysis in UTR or promoter, step S290 of copy-number variation analysis or a combination thereof corresponding to the variant type. In terms of variant types, compared to Sherloc rules, ACMG-AMP guidelines were considered too general, resulting in many variants getting more than deserved scores. Therefore, Sherloc mainly hoped to reduce probability of false positives, so classification is more rigorous on higher pathogenic scores. In practice, Holmes first input the VCF provided by the user into the VEP for execution, and then further scores with information provided by the VEP, such as the variant type and probability of loss of function intolerance (PLI). In this article, “probability of loss of function intolerance (PLI)” refers to degree of intolerance of a gene to loss-of-function (LOF) variants, which is calculated statistically by observing more than 60,000 variants in ExAc database, and the PLI value is between 0 and 1. The larger the value, the higher the risk when the gene loses its function. The general definition is >0.9 to be classified as severe. In some embodiments, optionally, it can be compared with the patient's family information and variant analysis, unrelated person information and variant analysis, disease, disease-free, historical analysis records, loss of function experimental data, etc. in step S01.

Please refer to FIG. 4, which illustrates a flowchart of a null variant analysis according to some embodiments of the present disclosure. The null variants defined by Sherloc refer to variants that may lose gene function. Sherloc defined a LOF mechanism by itself here. When a gene is “known” to lose its function and leads to serious consequences, the gene conforms to the LOF mechanism, and how serious is the result, Sherloc has its own definition, and it is input by the user whether it conforms to. However, this practice is a big hindrance to automation. Holmes disclosed in the present disclosure uses the PLI value to replace the LOF mechanism, which is conducive to the implementation of automation. In some embodiments, variant type data and a gene loss-of-function index are produced using the variant effect predictor based on the related information (e.g., variant sequence information (e.g., patient disease information and variant analysis, patient's family information and variant analysis, unrelated person information and variant analysis, disease, disease-free, historical analysis records), and loss of function experimental data). Step S210 of null variant analysis is performed according to the variant type data. Specifically,

in Step S211, Holmes then queries the ExAC_PLI value of the VEP, if it is greater than a predetermined threshold such as 0.9, it complies with the LOF mechanism of Sherloc and proceeds to step S2121; otherwise, it does not comply and proceeds to step S2122;

in steps S2121 and S2122, Holmes uses a search reference value (human reference, not mutated) to determine that the site belongs to which exon of the gene and where on the exon to determine whether it causes decay (nonsense-mediated mRNA decay, NMD). If the decay occurs, the corresponding evidence standard and score (i.e., the variant type score) can be obtained; if it does not occur, proceeds to the next step;

in steps S2131, S2132, and S2133, Holmes queries a pathogenic score of the judgment sites of the Clinvar (i.e., the variant type score).

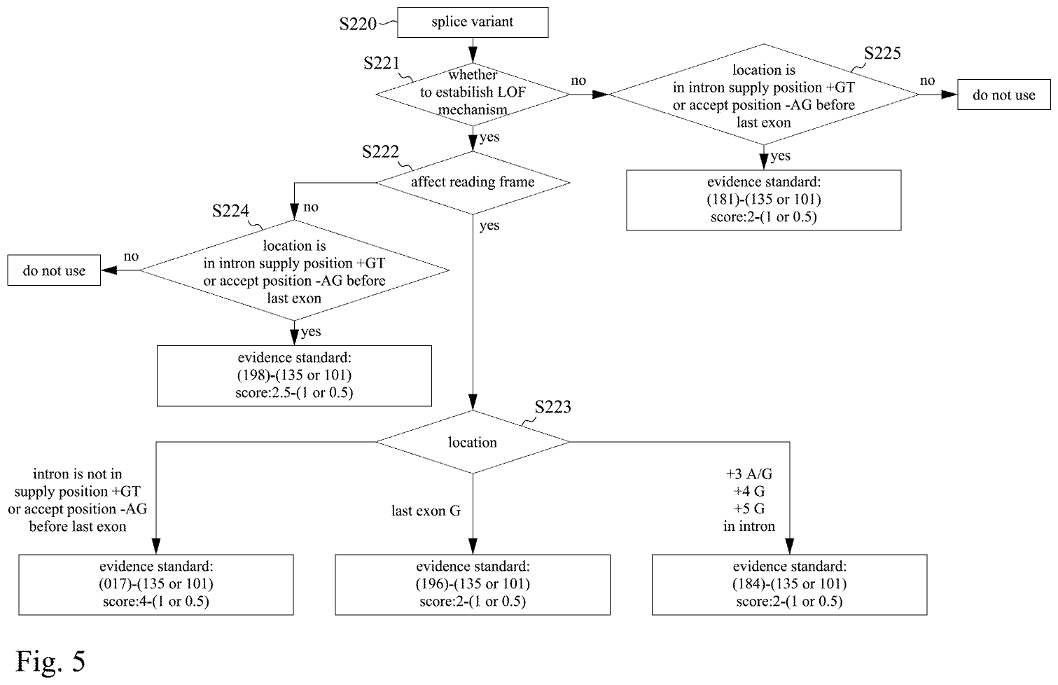

Please refer to FIG. 5, which illustrates a flowchart of a splice variant analysis according to some embodiments of the present disclosure. Sherloc believed that splice sites defined by ACMG-AMP guidelines were too general, and not all splice site variants affect splicing. Therefore, regarding the definition of the site of the splicing range, in addition to the more stringent position, there is also regulation for nucleotide change. In addition, the variant of the splice site is also affected by the LOF mechanism and has different score change. In some embodiments, the variant type data and the gene loss-of-function index are produced using the variant effect predictor based on the related information. According to the variant type data, step S220 of splicing variant analysis is performed. Specifically,

in step S221, Holmes then queries the ExAC_PLI value of the VEP, if it is greater than a predetermined threshold such as 0.9, it complies with the LOF mechanism of Sherloc and proceeds to step S222, otherwise, proceeds to step S225;

in step S222, Holmes observes whether the mutation insertion or indels is a multiple of 3 to determine whether it affects a reading frame, if yes, proceeds to step S223, if no, proceeds to step S224;

in step S223, Holmes can know the location of the mutation site in intron or exon by querying the reference data to determine the location, and obtain the corresponding evidence standard and score (i.e., the variant type score);

in step S224, Holmes can know the location of the mutation site in intron or exon by querying the reference data to determine whether the location is in the intron supply position +GT or accept position −AG before the last exon, if yes, the corresponding evidence standard and score (i.e., the variant type score) are obtained, if no, it is not used and the variant type score is 0;

in step S225, finally, Holmes can know the location of the mutation site in intron or exon by querying the reference data to judge whether the location is in the intron supply position +GT or accept position −AG before the last exon, if yes, the corresponding evidence standard and score (i.e., the variant type score) are obtained; if no, it is not used and the evidence standard and score (i.e., the variant type score) are 0.

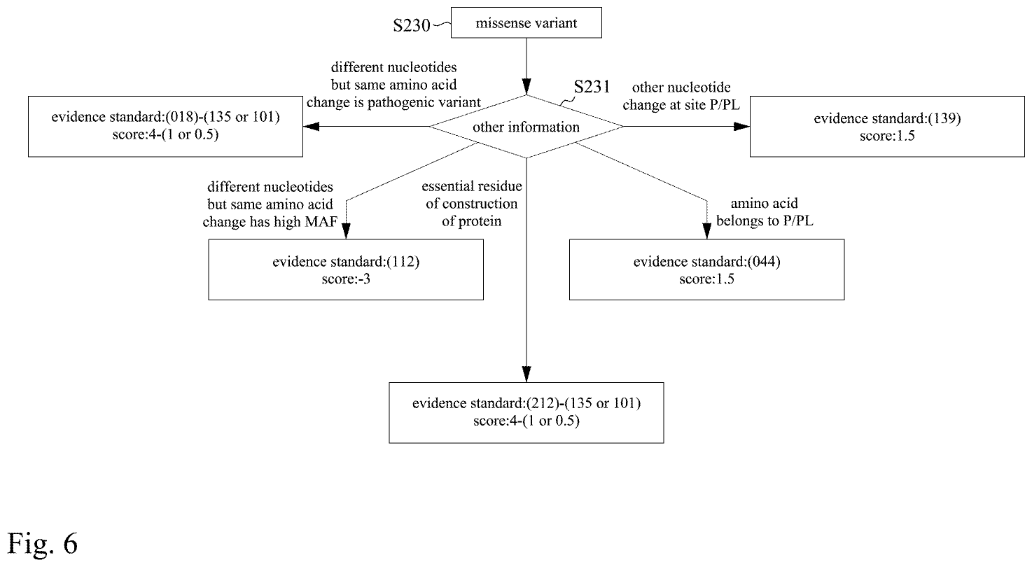

Please refer to FIG. 6, which illustrates a flowchart of a missense variant analysis according to some embodiments of the present disclosure. Sherloc rules believed that missense variants are not yet pathogenic, and must be classified as pathogenic with other information aids. In some embodiments, the variant type data and the gene loss-of-function index are produced using the variant effect predictor based on the related information. Step S230 of missense variant analysis is performed according to the variant type data. Specifically,

in step S231, Holmes can know amino acid change from the VEP, and can know pathogenic status of the site according to minor allele frequency (MAF) variant analysis obtained during step S10 of prevalence analysis and by automatically querying pathogenic information provided by the json file input by the user or automatically querying Clinvar. Combining the above evidences, the corresponding evidence standard and score (i.e., the variant type score) can be obtained when the evidence conditions are met.

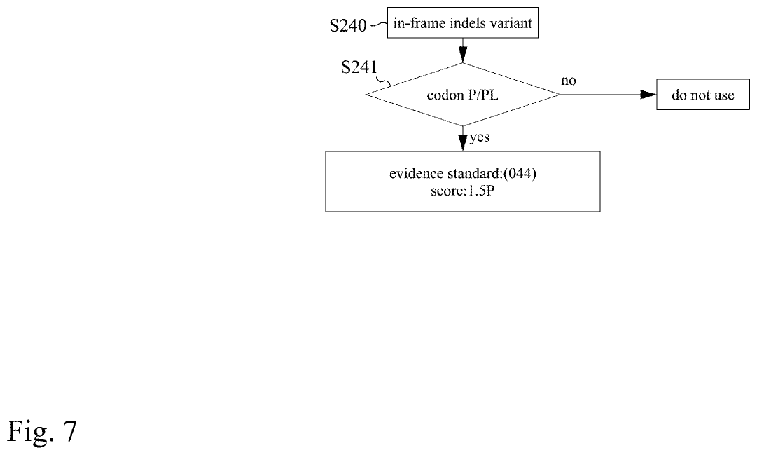

Please refer to FIG. 7, which illustrates a flowchart of an in-frame indels variant analysis according to some embodiments of the present disclosure. This case is usually classified as benign, and if insertion or indels is pathogenic, it is also judged to be pathogenic. In some embodiments, the variant type data and the gene loss-of-function index are produced using the variant effect predictor based on the related information. According to the variant type data, step S240 of in-frame indels variant analysis is performed. Specifically,

in step S241, Holmes can determine whether the site is a multiple of 3 by counting insertion or indels using the input VCF file, and can know whether the condon at the site is pathogenic or not (pathogenic (P)/likely pathogenic (LP)) by querying the disease information provided by the json file input by the user or automatically querying Clinvar. If it meets the evidence conditions, the corresponding evidence standard and score (i.e., the variant type score) can be obtained; otherwise, it is not used and the evidence standard and score (i.e., the variant type score) are 0.

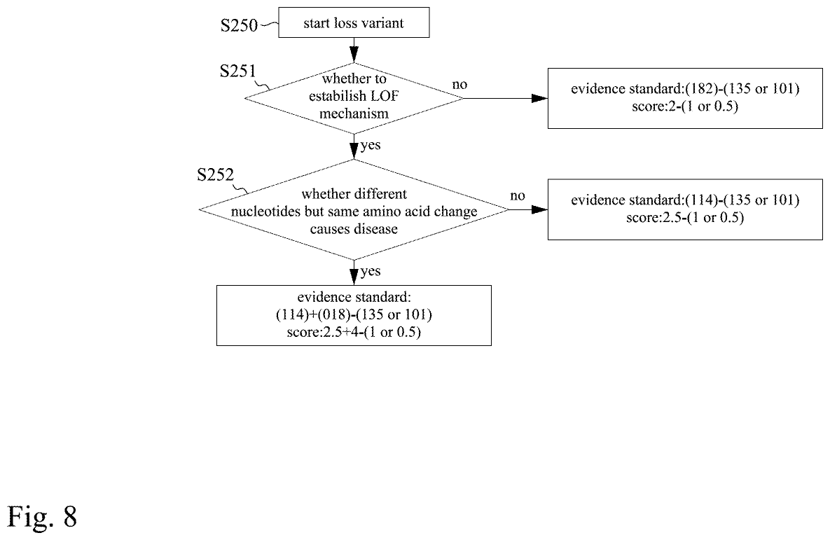

Please refer to FIG. 8, which illustrates a flowchart of a start loss variant analysis according to some embodiments of the present disclosure. Sherloc believed that in this case, the LOF mechanism and whether this case is recorded as pathogenic as classification should be considered. In some embodiments, the variant type data and the gene loss-of-function index are produced using the variant effect predictor based on the related information. According to the variant type data, step S250 of start loss variant analysis is performed. Specifically,

in step S251, Holmes then queries the ExAC_PLI value of the VEP, if it is greater than a predetermined threshold such as 0.9, it complies with the LOF mechanism of Sherloc and proceeds to step S252, otherwise the corresponding evidence standard and score (i.e., the variant type score) are obtained;

in step S252, Holmes can know pathogenic status of the site by querying the disease information provided by the json file input by the user or automatically querying Clinvar, and Holmes combines results of Clinvar according to the amino acid change of the VEP to obtain the corresponding evidence standard and score (i.e., the variant type score).

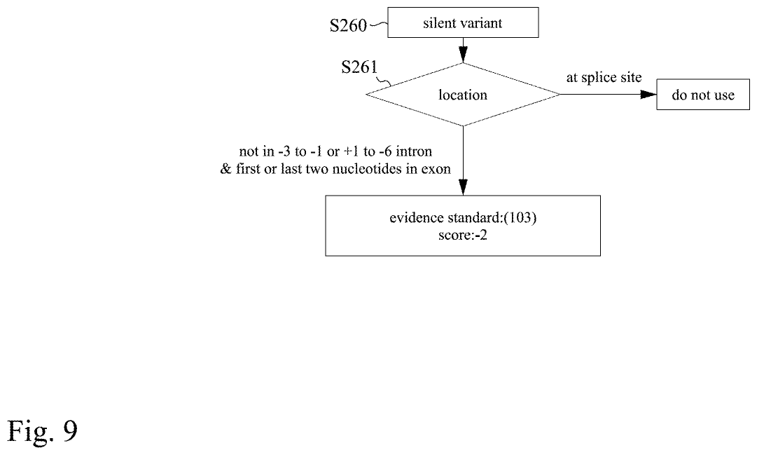

Please refer to FIG. 9, which illustrates a flowchart of a silent variant analysis according to some embodiments of the present disclosure. In general, this case is classified as benign, and Sherloc also classified it as benign if it avoided the splice site. In some embodiments, the variant type data and the gene loss-of-function index are produced using the variant effect predictor based on the related information. According to the variant type data, step S260 of silent variant analysis is performed. Specifically,

in step S261, Holmes can know whether the site belongs to the splice site mutation defined by Sherloc rules by querying the reference data according to the site. If it is at the splice site, it is not used and the score (i.e., the variant type score) is 0; otherwise, the corresponding evidence standard and score (i.e., the variant type score) are obtained.

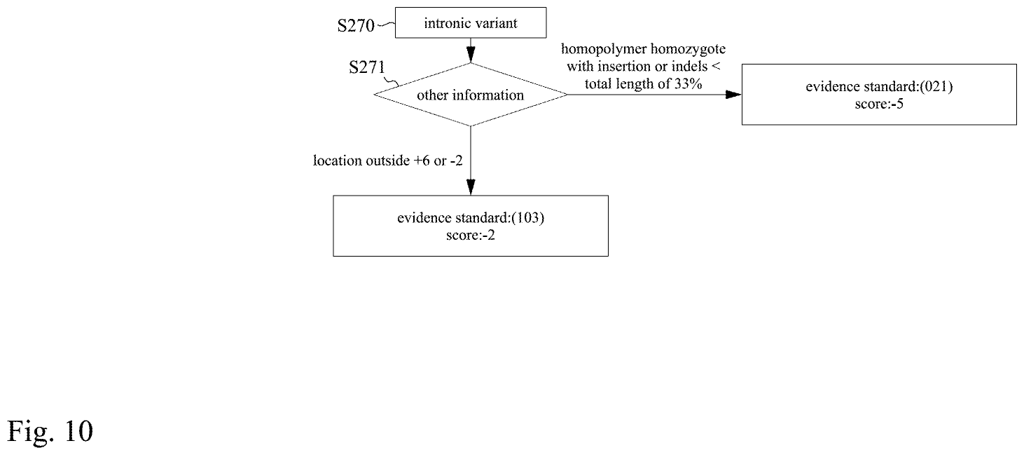

Please refer to FIG. 10, which illustrates a flowchart of an intronic variant analysis according to some embodiments of the present disclosure. In general, this case is classified as benign, and Sherloc believed that if the insertion or indels was too long, probability of being benign was reduced. In some embodiments, the variant type data and the gene loss-of-function index are produced using the variant effect predictor based on the related information. According to the variant type data, step S270 of intronic variant analysis is performed. Specifically,

in step S271, Holmes can obtain a total length of the insertion or indels by querying the reference data and automatically analyzing the VCF of the json file input by the user, or Holmes can also know the location of the insertion or indels in intron by querying the reference data to obtain the corresponding evidence standard and score (i.e., the variant type score).

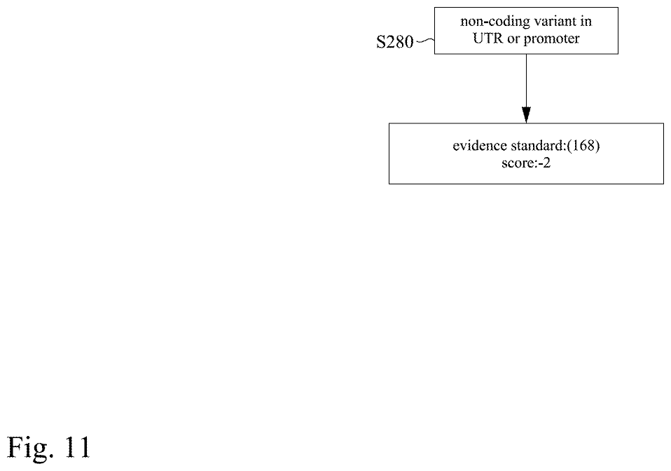

Please refer to FIG. 11, which illustrates a flowchart of a non-coding variant analysis in UTR or promoter according to some embodiments of the present disclosure. In general, this case is classified as benign, which is directly judged by the variant type provided by the VEP. In some embodiments, the variant type data and the gene loss-of-function index are produced using the variant effect predictor based on the related information. According to the variant type data, step S280 of non-coding variant analysis in UTR or promoter is performed. Specifically, Holmes determines that if the variant type of the VEP does not belong to any of the above variant types, it is classified as this, and the corresponding evidence standard and score (i.e., the variant type score) are obtained.

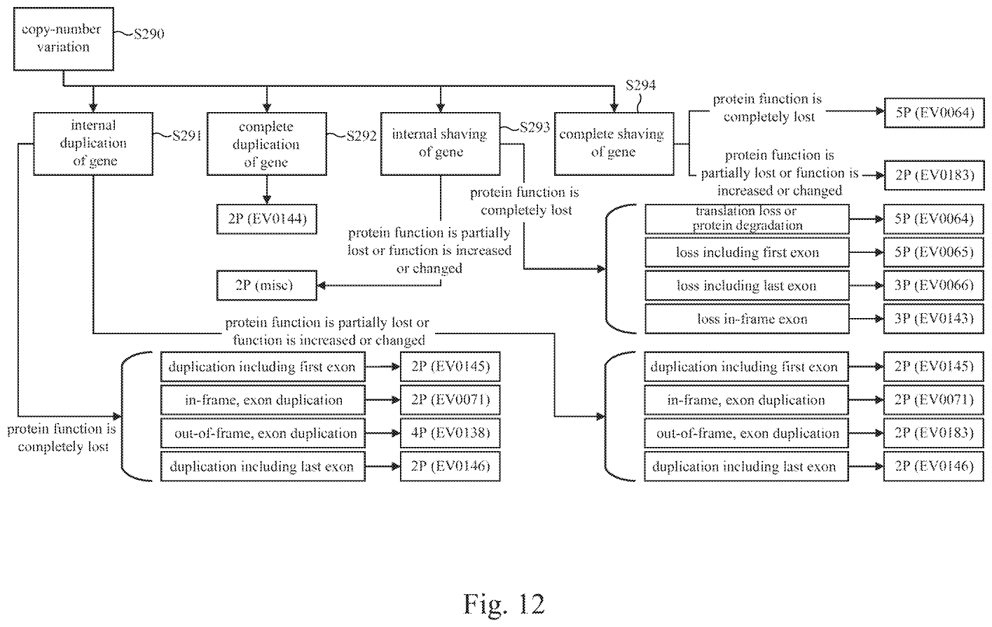

Please refer to FIG. 12, which illustrates a flowchart of a copy-number variation analysis according to some embodiments of the present disclosure. According to the variant type data, step S290 of copy-number variation analysis is performed. The copy-number variation is supposed to be fairly rare, and if it is a frequent variant, it should be considered as another variant type. If shaving in the copy-number variation occurs at known splice variant, the splice variant should be used. Specifically, step S290 of copy-number variation distinguishing gene type of copy-number variation proceeds to steps S291 to S294 respectively:

in step S291, internal duplication of gene, if protein function is completely lost, it is classified as (1) duplication including the first exon, evidence standard is 145, and evidence score is 2; (2) in-frame, exon duplication, evidence standard is 71, and evidence score is 2; (3) out-of-frame, exon duplication, evidence standard is 138, and evidence score is 4; (4) duplication including the last exon, evidence standard is 146, and evidence score is 2. If protein function is partially lost or the function is increased or changed, it is classified as (1) duplication including the first exon, evidence standard is 145, and evidence score is 2; (2) in-frame, exon duplication, evidence standard is 71, and evidence score is 2; (3) out-of-frame, exon duplication, evidence standard is 138, and evidence score is 2; (4) duplication including the last exon, evidence standard is 146, and evidence score is 2.

In step S292, complete duplication of gene, evidence standard is 144, and evidence score is 2.

In step S293, internal shaving of gene, if protein function is partially lost or the function is increased or changed, the evidence standard is misc, and evidence score is 2; if protein function is completely lost, it is classified as (1) translation loss or protein degradation, evidence standard is 64, and evidence score is 5; (2) loss including the first exon, evidence standard is 65, and evidence score is 5; (3) loss including the last exon, evidence standard is 66, and evidence score is 3; (4) loss in-frame exon, evidence standard is 143, and evidence score is 3.

In step S294, complete shaving of gene, if protein function is completely lost, evidence standard is 64, and evidence score is 5; if protein function is partially lost or the function is increased or changed, evidence standard is 183, and evidence score is 2.

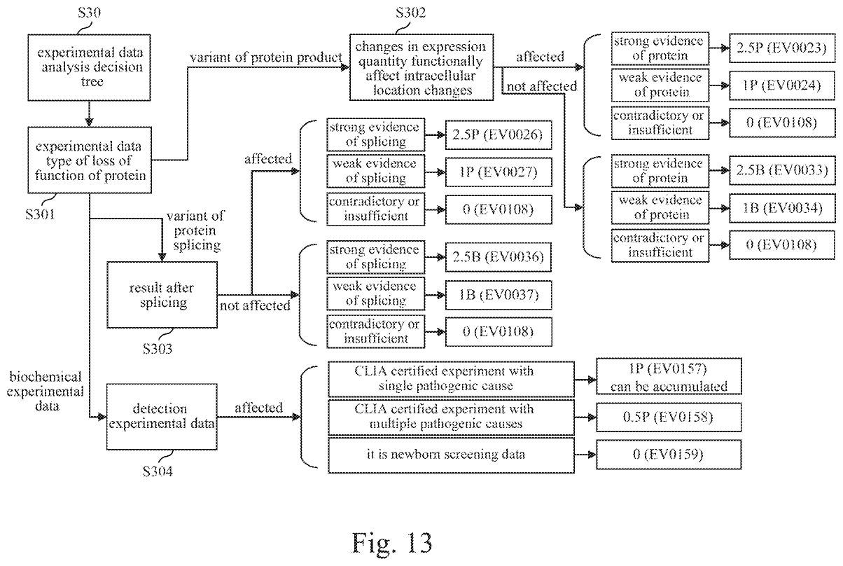

Please refer to FIG. 13, which illustrates a flowchart of an experimental data analysis according to some embodiments of the present disclosure. When it is determined in step S25 that the variant causes loss of function of the gene, proceeds to step S30 of experimental data analysis, whether it is caused by gene defect. Specifically,

in step S301, experimental data type of loss of function of protein, if it is a variant of a protein product, proceeds to step S302; if it is a variant of protein splicing, proceeds to step S303; if it is biochemical experimental data (e.g., protease chemical reaction detection, etc.), proceeds to step S304.

In step S302, changes in expression quantity functionally affect intracellular location changes. If it is affected, it is classified as (1) strong evidence of protein (i.e., protein function has been completely lost, or there are three or more weak protein experimental data), evidence standard is 23, and evidence score is 2.5; (2) weak evidence of protein (i.e., protein function has not been lost and part of function remains, or only a single or part of protein experimental data), evidence standard is 24, and evidence score is 1; (3) contradictory or insufficient, evidence standard is 108, and evidence score is 0. If it is not affected, it is classified as (1) strong evidence of protein, evidence standard is 33, and evidence score is 2.5; (2) weak evidence of protein, evidence standard is 34, and evidence score is 1; (3) contradictory or insufficient, evidence standard is 108, and evidence score is 0.

In step S303, the result after splicing. If it is affected, it is classified as (1) strong evidence of splicing (i.e., incidence of heterozygotes is close to 50%, or incidence of homozygotes is close to 100%, and causes near or complete loss of function of protein), evidence standard is 26, and evidence score is 2.5; (2) weak evidence of splicing (i.e., after splice variant, skipping a complete exon without causing translational frame shifts, or the sampled tissue is an unaffected tissue), evidence standard is 27, and evidence score is 1; (3) contradictory or insufficient, evidence standard is 108, and evidence score is 0. If it is not affected, it is classified as (1) strong evidence of splicing, evidence standard is 36, and evidence score is 2.5; (2) weak evidence of splicing, evidence standard is 37, and evidence score is 1; (3) contradictory or insufficient, evidence standard is 108, and evidence score is 0.

In step S304, detection experimental data is compared with protease kinetic chemical analysis data provided in step S01. If it is affected, it is classified as (1) the Clinical Laboratory Improvement Amendments (CLIA) certified experiment with a single pathogenic cause, evidence standard is 157, and evidence score is 1; (2) the CLIA certified experiment with multiple pathogenic causes, evidence standard is 158, and evidence score is 0.5; (3) it is newborn screening data, evidence standard is 159, and evidence score is 0.

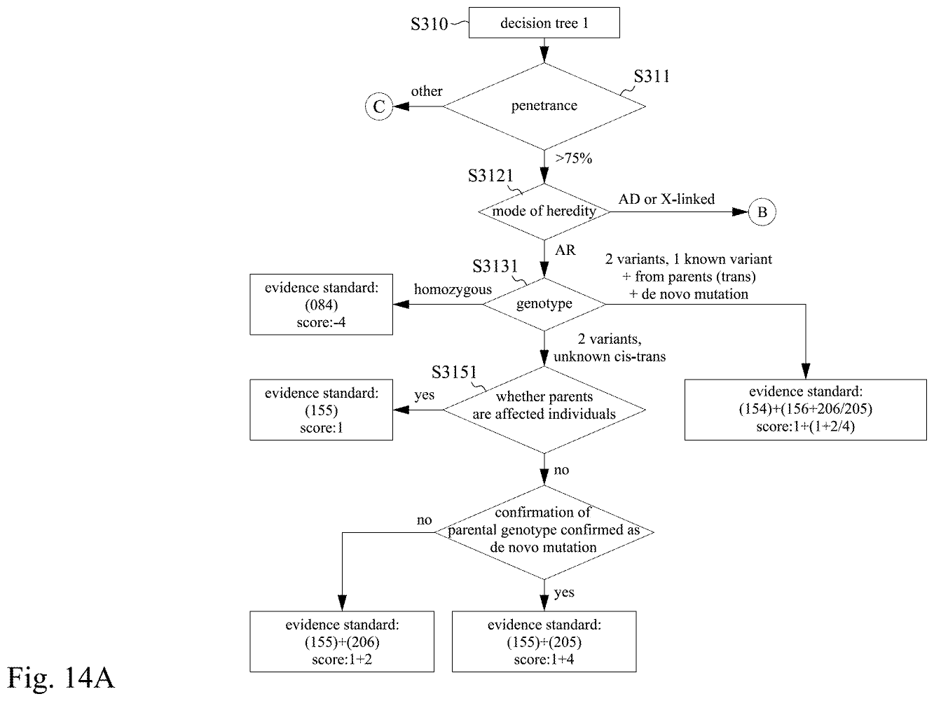

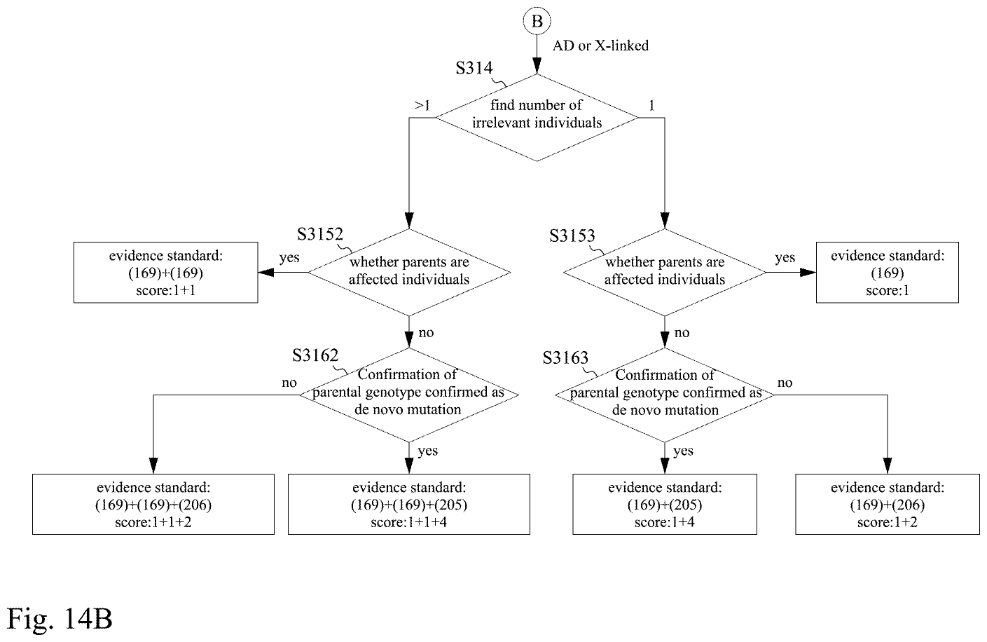

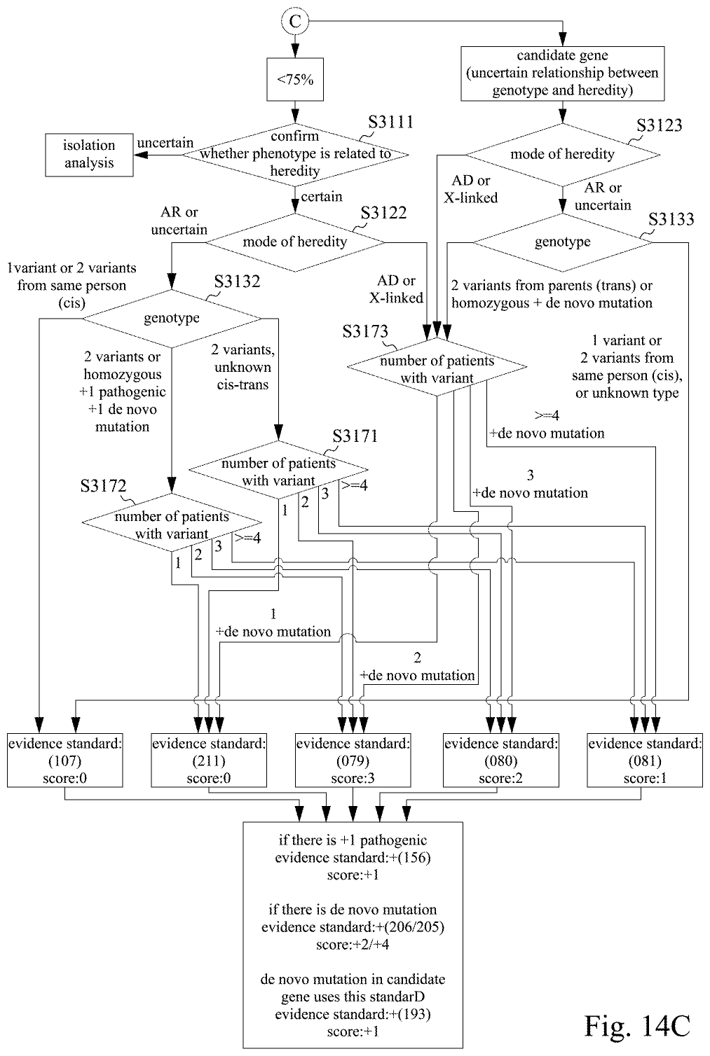

Please refer to FIG. 14A to FIG. 16, step S35 of clinical data comparison, Sherloc believed that in the past guidelines, clinical data is always the most neglected part, but clinical information is the data that is most relevant to the patient and the disease, so when Sherloc rules were developed, clinical data was given a fairly high pathogenic score. When clinical data conflicts with database or predicted data, information of clinical data will be given priority.

Sherloc clinical rules first classified whether the subject is sick or not, and degree of mastery of the disease. If the subject is a healthy person, it is classified as decision tree 3; if it is a patient, based on knowledge of the disease. If we know the disease is caused by another reason, it is classified as decision tree 2; if pathogenic cause is unknown and proportion of population is low, it is classified as decision tree 1. Specifically,

1. Holmes first determines whether the subject is a patient. The judgment method is to automatically query the json file data input by the user. If yes, proceeds to the next step, otherwise proceeds to step S320 of decision tree 3.

2. If genotype is consistent with phenotype, proceeds to the next step, otherwise it is not used, and fixed to yes on use.

3. Whether the disease has another pathogenic cause, and fixed to no on use, if the pathogenic cause is known, proceeds to step S330 of decision tree 2.

4. Proceeds to step S310 of decision tree 1.

Please refer to FIG. 14A to FIG. 14C. FIG. 14A is a flowchart of clinical data comparison of a patient with unknown pathogenic cause according to some embodiments of the present disclosure. FIG. 14B shows a flowchart of the process B of FIG. 14A. FIG. 14C shows a flowchart of the process C of FIG. 14A. When the subject is a patient and meets rules of decision tree 1, the analysis continues to the lower level. First, in decision tree 1, penetrance of the disease is classified, and then different pathogenic scores are given according to dominant-recessive gene, genotype, cis-trans, and whether it is a de novo mutation. If penetrance is less than 75% or uncertain, additional consideration is given to a number of patients with the same disease at the same site due to greater uncertainty. In the part of familial diseases, Sherloc further defines an isolation analysis. If the user has provided information of relatives, it can obtain the score of the isolation analysis by searching whether the site of the VCF of the relatives is mutated. In some embodiments, the step of producing a clinical score using the related information or the clinical database based on the related information includes judging whether the subject is a patient based on the related information, and then performing dominant-recessive analysis, genotype analysis, cis-trans analysis, disease penetrance analysis, age of onset analysis, or a combination thereof. Step S310 of decision tree 1, specifically,

in step S311, Holmes automatically queries the json file input by the user to determine penetrance, which is classified as more than 75%, less than 75% and uncertain; if it is more than 75%, proceeds to step S3121; if it is less than 75%, proceeds to step S3111; and if it is uncertain, proceeds to step S3123.

In step S3111, Holmes then automatically queries other patient data of the json file input by the user to determine whether the phenotype is related to genetics. If yes, proceeds to step S3122, otherwise proceeds to isolation analysis;

in steps S3121, S3122 and S3123, Holmes automatically queries the json file input by the user or automatically queries Clinvar to judge gene dominant and recessive such as autosomal recessive (AR), autosomal dominant (AD) or X-linked, and recessive gene proceeds to steps S3131, S3132 and S3133, respectively, otherwise proceeds to steps S314, S3173, and S3173, respectively;

in steps S3131, S3132, and S3133, Holmes judges cis-trans by automatically querying parental data in the json file input by the user. The two nucleotides at the same site respectively from father and mother are in trans, or those from the same person are in cis. In step S3131, when the genotype is 2 variants and unknown cis-trans, proceeds to step S3151; when the genotype is homozygous, or 2 variants, or 1 known variant and trans and de novo mutation, the corresponding evidence standard and score (i.e., the clinical score) can be obtained; in step S3132, when the genotype is 1 variant or 2 variants cis, the corresponding evidence standard and score (i.e., the clinical score) are obtained; when the genotype is 2 variants and unknown cis-trans, proceeds to step S3171; when the genotype is (2 variants or homozygous)+1 pathogenic+1 de novo mutation, proceeds to step S3172; in step S3133, when the genotype is (2 variants cis or homozygous)+de novo mutation, proceeds to step S3173; when the genotype is 1 variant or 2 variants cis, or the genotype is unknown, the corresponding evidence standard and score (i.e., the clinical score) are obtained;

in step S314, Holmes finds a number of irrelevant individuals, if it is greater than 1, proceeds to step S3152, if it is equal to 1, proceeds to step S3153;

in steps S3151, S3152, S3153, Holmes judges whether the parents are affected individuals, if yes, the corresponding evidence standard and score (i.e., the clinical score) are obtained, if no, proceeds to steps S3161, S3162, and S3163, respectively;

in steps S3161, S3162, and S3163, Holmes queries parental data in the json file data input by the user to determine whether it is the site of the de novo mutation. According to yes or no, the corresponding evidence standard and score (i.e., the clinical score) are obtained;

in steps S3171, S3172, and S3173, Holmes determines a number of patients with the variant, and the corresponding evidence standard and score (i.e., the clinical score) are obtained.

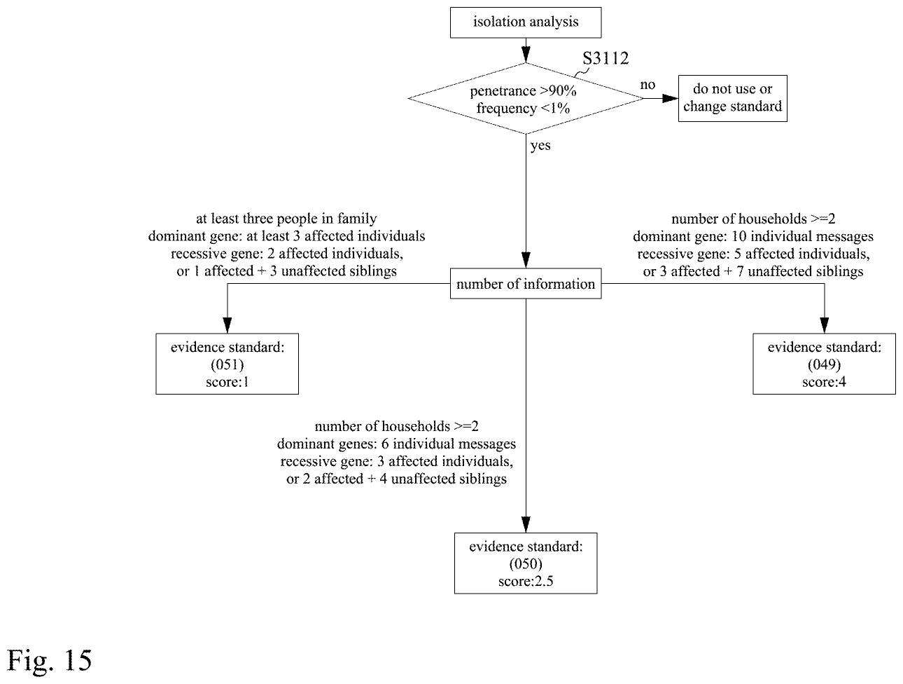

Please refer to FIG. 15, which shows a flowchart of the isolation analysis of FIG. 14C. Specifically,

in step S3112, Holmes queries gnomAD to confirm whether frequency of the site is less than 1%, and Holmes confirms whether penetrance in the json file input by the user is greater than 90%. If no, it is not used or changes the standard; if Holmes queries the relative data provided by the json file input by the user, confirms a number and health status of the relatives with the variant, and the corresponding evidence standard and score (i.e., the clinical score) are obtained.

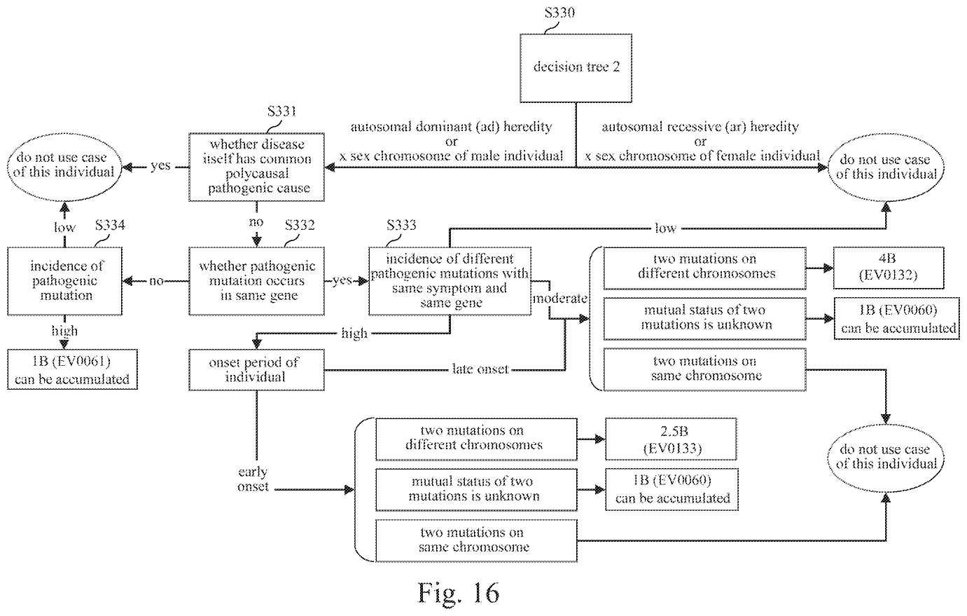

Please refer to FIG. 16, which shows a flowchart of clinical data comparison of patients with known pathogenic cause according to some embodiments of the present disclosure, which is used for comparison with the specific target disease or gene selection provided in step S01. When the subject is a patient and meets rules of decision tree 2 (judgment of cases with different variants and same symptoms), the analysis continues to the lower level. Step S330 of decision tree 2, specifically,

If it is autosomal recessive (AR) inheritance or an X sex chromosome of a female individual, the case of the individual is not used; if it is autosomal dominant (AD) inheritance or an X sex chromosome of a male individual, proceeds to step S331.

Step S331 determines whether the disease itself has common polycausal pathogenic cause, and if yes, the case of the individual is not used; if no, proceeds to step S332.

Step S332 determines whether pathogenic mutation occurs in the same gene. If yes, proceeds to step S333; if no, proceeds to step S334.

Step S333 determines incidence of different pathogenic mutations with same symptoms and same gene. If incidence is low, the case of the individual is not used. If incidence is moderate, it is classified as: (1) two mutations on different chromosomes, evidence standard is 132, and evidence score is 4; (2) mutual status of two mutations is unknown, and evidence standard is 60, and evidence score is 1; (3) two mutations on same chromosome, the case of the individual is not used. If incidence is high, the individual onset period needs to be further judged: if it is early onset, it is classified as (1) two mutations on different chromosomes, evidence standard is 133, and evidence score is 2.5; (2) mutual status of two mutations is unknown, evidence standard is 60, and evidence score is 1; (3) two mutations on same chromosome, the case of the individual is not used. In case of late onset, those are the same as the classification and scoring method of the moderate incidence.

Step S334 determines incidence of pathogenic mutation, if incidence is high, evidence standard is 61, and evidence score is 1; if incidence is low, the case of the individual is not used.

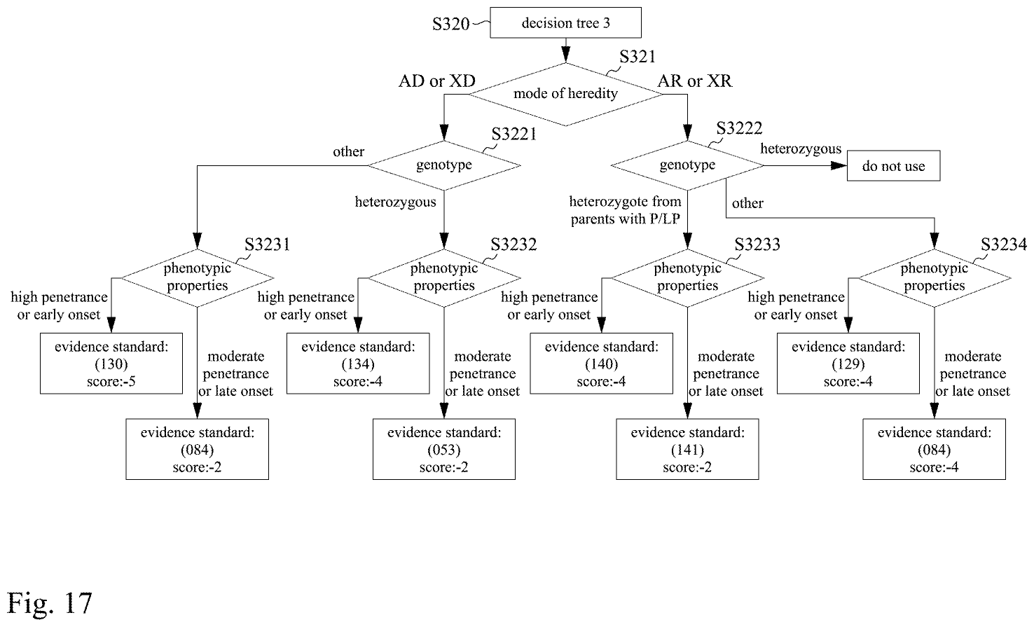

Please refer to FIG. 17, which illustrates a flowchart of clinical data comparison of a healthy subject according to some embodiments of the present disclosure. When the subject is a healthy person, Sherloc rules proceeds to decision tree 3 at this time. This part is also determined by dominant recessive, genotype, cis-trans, disease penetrance, and age of onset. Step S320 of decision tree 3, specifically,

in step S321, Holmes obtains gene dominant-recessive (e.g., AD; AR; X-chromosome dominant, X-linked dominant (XD); X-chromosome recessive, X-linked recessive (XR)). If it is AD or XD, proceeds to step S3221, if it is AR or XR, proceeds to step S3222;

in steps S3221 and S3222, Holmes determines genotype of cis-trans using parental data in the json file input by the user. In step S3221, if it is homozygous, proceeds to step S3232; if it is other, proceeds to step S3231. In step S3222, if it is heterozygous, it is not used; if heterozygous is cis and is P/LP, proceeds to step S3233; if it is other, proceeds to step S3234.

In steps S3231, S3232, S3233, and S3234, Holmes obtains disease penetrance and age of onset through the json file input by the user or by automatically querying Clinvar, and combines the above information to obtain the corresponding evidence standard and score (i.e., the clinical score).

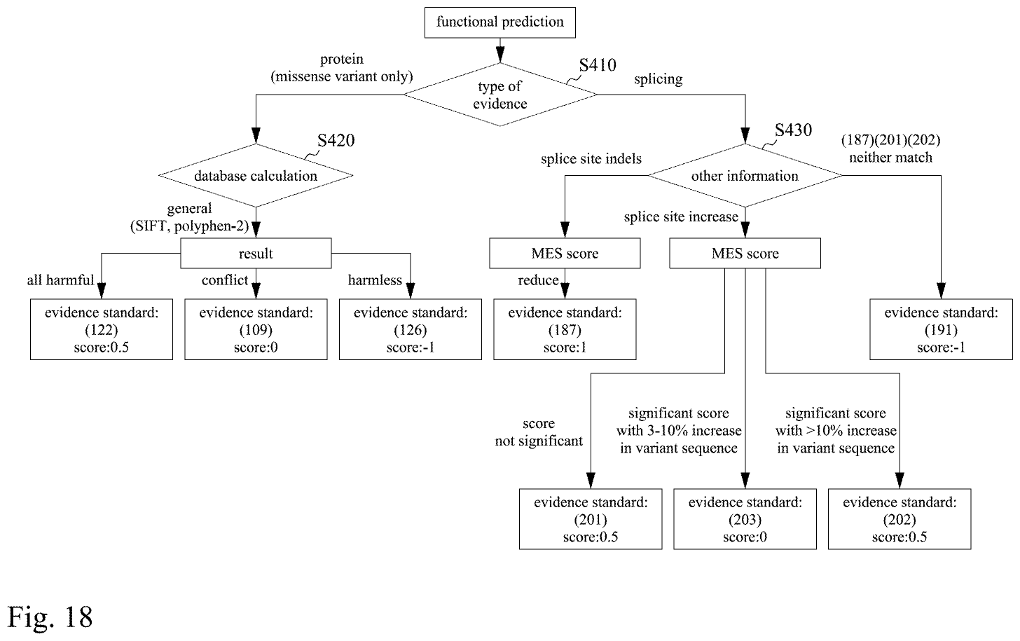

Please refer to FIG. 18, which illustrates a flowchart of a functional predictive analysis according to some embodiments of the present disclosure. Step S40 is computational simulation analysis, which uses prediction results of other tools for analysis. The analysis is classified as two levels. The first is whether it affects a protein product. This part is limited to missense analysis; the second is the effect of splicing. In some embodiments, the step of producing a functional score using a functional variant hazard prediction tool based on the related information includes determining whether the mutation sites in the variant sequence information of the related information are missense variant or splicing variant. In some embodiments, the functional variant hazard prediction tool includes, but is not limited to, the VEP judgments. Specifically,

in step S410, Holmes determines type of evidence. If the mutation site is a missense variant, proceeds to step S420; if it is a splicing variant, proceeds to step S430.

In step S420, Holmes uses built-in SIFT and polymorphism phenotype analysis (e.g., polyphen-2) of the VEP to obtain the corresponding evidence standard and score (i.e., the functional score).

In step S430, Holmes uses the site hazard prediction (e.g., MES) of the VEP plug-in built in the VEP to obtain the corresponding evidence standard and score (i.e., the functional score).

The above only exemplifies execution of some of Sherloc rules, and other rules can also be fully automated by the same concept, which will not be repeated here.

In some embodiments of the present disclosure, semi-automated Sherloc rules is simplified by using the PLI value instead of the LOF mechanism to achieve full automation without losing accuracy. The final scoring method is to set various standard scores. In the future, the standard can be expanded by modifying the pathogenic (benign) score threshold.

In some embodiments of the present disclosure, Helmes uses updated and more accurate rules to implement fully automated implementation, and the user only needs to prepare the data to use, and the rules do not have to judge by themselves, which will lower the use threshold and save a lot of human interpretation time.

Although the present disclosure has been disclosed in the above embodiments, it is not intended to limit the present disclosure. Anyone who is familiar with this technique can make various changes and modifications without departing from the spirit and scope of the present disclosure. Therefore, the scope of protection of the present disclosure shall be subject to the scope of appended claims.

What is claimed is:

1. An automated pathogenic mutation classification method, comprising:
receiving related information, the related information including variant sequence information, the variant sequence information including patient information and variant analysis, patient's family information and variant analysis, or unrelated person information and variant analysis;
producing a population score using a population database based on the related information;
producing a variant type score using a variation pattern prediction tool based on the related information;
producing a clinical score using the related information or a clinical database based on the related information, wherein the clinical database includes Clinvar database;
producing a functional score using a functional variant hazard prediction tool based on the related information;
summing the population score, the variant type score, the clinical score, and the functional score to produce a pathogenic score; and
determining probability that a plurality of mutation sites in the variant sequence information suffer from a corresponding disease based on the pathogenic score, wherein when the pathogenic score is higher, the probability of the mutation sites suffering from the corresponding disease is higher.

2. The classification method of claim 1, wherein the related information further comprises loss-of-function test data, protease kinetic chemical analysis data, special target disease or gene selection, or a combination thereof.

3. The classification method of claim 1, wherein the step of using the population database comprises:
performing a frequency variation analysis on the variant sequence information using the population database to generate a first population score;
performing a homozygous observational analysis on the variant sequence information using the population database to generate a second population score; and
summing the first population score and the second population score to obtain the population score.

4. The classification method of claim 3, wherein the population database comprises a genome aggregation database and a 1,000 genomes project database, wherein the step of performing the frequency variation analysis on the variant sequence information using the population database,
when the mutation sites in the variant sequence information in a plurality of alleles in the genome aggregation database are greater than a predetermined threshold number, continue to use the genome aggregation database to perform the frequency variation analysis; or
when the mutation sites in the variant sequence information in the alleles in the genome aggregation database are less than or equal to the predetermined threshold number, use the 1,000 genomes project database to perform the frequency variation analysis.

5. The classification method of claim 3, wherein the step of performing the homozygous observational analysis on the variant sequence information using the population database,
when the mutation sites in the variant sequence information in a plurality of alleles in the genome aggregation database are greater than a predetermined threshold number, continue to use the population database to perform the homozygous observational analysis; or
when the mutation sites in the variant sequence information in the alleles in the genome aggregation database are less than or equal to the predetermined threshold number, the homozygous observational analysis is not performed.

6. The classification method of claim 1, wherein the step of producing the variant type score using the variation pattern prediction tool based on the related information comprises:
producing gene sequence variation hazard information using the variation pattern prediction tool based on the related information; the gene sequence variation hazard information including variant type data and a gene loss-of-function index; and
performing a null variant analysis, a splice variant analysis, a missense variant analysis, an in-frame indels variant analysis, a start loss variant analysis, a silent variant analysis, an intronic variant analysis, a non-coding variant analysis in UTR or promoter, a copy-number variation analysis, or a combination thereof based on the variant type data to obtain the variant type score.

7. The classification method of claim 6, wherein the gene loss-of-function index is probability of loss of function intolerance.

8. The classification method of claim 7, wherein when the null variant analysis, the splice variant analysis, and the start loss variant analysis are performed to evaluate the probability of loss of function intolerance,
if the probability of loss of function intolerance is greater than a predetermined threshold, it automatically determines that a risk is high when one or more genes in the related information are loss of function; or
if the probability of loss of function intolerance is less than the predetermined threshold, it automatically determines that a risk is low when one or more genes in the related information are loss of function.

9. The classification method of claim 1, wherein the step of producing the clinical score using the related information or a clinical database based on the related information comprises judging whether it is a patient based on the related information, and then performing a dominant-recessive analysis, a genotype analysis, a cis-trans analysis, a disease penetrance analysis, an age of onset analysis, or a combination thereof.

10. The classification method of claim 1,
wherein the functional variant hazard prediction tool comprises a scale-invariant feature transform unit, a polymorphism phenotype analysis unit, and a site hazard prediction unit;
wherein the step of producing the functional score using the functional variant hazard prediction tool based on the related information comprises judging whether the mutation sites in the variant sequence information of the related information are a missense variant or a splicing variant,
when the mutation sites are the missense variant, analysis with the scale-invariant feature transform unit and the polymorphism phenotype analysis unit is performed to produce the functional score; or
when the mutation sites are the splicing variant, analysis with the site hazard prediction unit is performed to produce the functional score.

11. An automated pathogenic mutation classifier, comprising a computer processor and a memory, the memory storing a plurality of computer program instructions that, when executed by the computer processor, cause the computer processor to implement following steps, comprising:
accessing related information, the related information including variant sequence information, the variant sequence information including patient information and variant analysis, patient's family information and variant analysis, or unrelated person information and variant analysis;
producing a population score using a population database based on the related information;
producing a variant type score using a variation pattern prediction tool based on the related information;
producing a clinical score using the related information or a clinical database based on the related information, wherein the clinical database includes Clinvar database;
producing a functional score using a functional variant hazard prediction tool based on the related information;
summing the population score, the variant type score, the clinical score, and the functional score to produce a pathogenic score; and
determining probability that a plurality of mutation sites in the variant sequence information suffer from a corresponding disease based on the pathogenic score, wherein when the pathogenic score is higher, the probability of the mutation sites suffering from the corresponding disease is higher.

12. The classifier of claim 11, wherein the related information further comprises loss-of-function test data, protease kinetic chemical analysis data, special target disease or gene selection, or a combination thereof.

13. The classifier of claim 11, wherein the step of using the population database comprises:
performing a frequency variation analysis on the variant sequence information using the population database to generate a first population score;
performing a homozygous observational analysis on the variant sequence information using the population database to generate a second population score; and
summing the first population score and the second population score to obtain the population score.

14. The classifier of claim 13, wherein the population database comprises genome aggregation database and 1,000 genomes project database, wherein the step of performing the frequency variation analysis on the variant sequence information using the population database,
when the mutation sites in the variant sequence information in a plurality of alleles in the genome aggregation database are greater than a predetermined threshold number, continue to use the genome aggregation database to perform the frequency variation analysis; or
when the mutation sites in the variant sequence information in the alleles in the genome aggregation database are less than or equal to the predetermined threshold number, use the 1,000 genomes project database to perform the frequency variation analysis.

15. The classifier of claim 13, wherein the step of performing the homozygous observational analysis on the variant sequence information using the population database,
when the mutation sites in the variant sequence information in a plurality of alleles in the genome aggregation database are greater than a predetermined threshold number, continue to use the population database to perform the homozygous observational analysis; or
when the mutation sites in the variant sequence information in the alleles in the genome aggregation database are less than or equal to the predetermined threshold number, the homozygous observational analysis is not performed.

16. The classifier of claim 11, wherein the step of producing the variant type score using the variation pattern prediction tool based on the related information comprises:
producing gene sequence variation hazard information using the variation pattern prediction tool based on the related information; the gene sequence variation hazard information including variant type data and a gene loss-of-function index; and
performing a null variant analysis, a splice variant analysis, a missense variant analysis, an in-frame indels variant analysis, a start loss variant analysis, a silent variant analysis, an intronic variant analysis, a non-coding variant analysis in UTR or promoter, a copy-number variation analysis, or a combination thereof based on the variant type data to obtain the variant type score.

17. The classifier of claim 16, wherein the gene loss-of-function index is probability of loss of function intolerance.

18. The classifier of claim 17, wherein when the null variant analysis, the splice variant analysis, and the start loss variant analysis are performed to evaluate the probability of loss of function intolerance,
if the probability of loss of function intolerance is greater than a predetermined threshold, it automatically determines that a risk is high when one or more genes in the related information are loss of function; or
if the probability of loss of function intolerance is less than the predetermined threshold, it automatically determines that a risk is low when one or more genes in the related information are loss of function.

19. The classifier of claim 11, wherein the step of producing the clinical score using the related information or a clinical database based on the related information comprises judging whether it is a patient based on the related information, and then performing a dominant-recessive analysis, a genotype analysis, a cis-trans analysis, a disease penetrance analysis, an age of onset analysis, or a combination thereof.

20. The classifier of claim 11,
wherein the functional variant hazard prediction tool comprises a scale-invariant feature transform unit, a polymorphism phenotype analysis unit, and a site hazard prediction unit;
wherein the step of producing the functional score using the functional variant hazard prediction tool based on the related information comprises judging whether the mutation sites in the variant sequence information of the related information are a missense variant or a splicing variant,
when the mutation sites are the missense variant, analysis with the scale-invariant feature transform unit and the polymorphism phenotype analysis unit is performed to produce the functional score; or
when the mutation sites are the splicing variant, analysis with the site hazard prediction unit is performed to produce the functional score.
